(19) United States
(12) Patent Application Publication
(10) Pub. No.: US 2017/0010258 A1
(43) Pub. Date: Jan. 12, 2017

(54) METAL-INSULATOR TRANSITION POINT BIOSENSOR

(71) Applicant: BOARD OF TRUSTEES OF THE LELAND STANFORD JUNIOR UNIVERSITY, Palo Alto, CA (US)

(72) Inventors: Ronald W. Davis, Palo Alto, CA (US); Rahim Esfandyarpour, Mountain View, CA (US); James S. Harris, Stanford, CA (US)

(21) Appl. No.: 15/119,468

(22) PCT Filed: Feb. 20, 2015
(86) PCT No.: PCT/US15/16762

§ 371 Date: Aug. 17, 2016

Related U.S. Application Data
(60) Provisional application No. 61/943,015, filed on Feb. 21, 2014.

(51) Int. Cl. G01N 33/543; G01N 27/12

(52) U.S. Cl. CPC G01N 33/5438; G01N 27/125

(57) ABSTRACT

The invention relates to a novel biosensor, the metal-insulator transition (MIT) point biosensor, a non-expensive miniaturized device, having a small footprint and high sensitivity, which can measure molecular interactions or the presence of small amounts of molecules without the need for the molecules to be labeled. The sensor comprises a vanadium dioxide (V02) layer located between two metal measuring pads. The introduction of molecules of interest to the sensor surface results in changes in the oxide interface charge density that can be detected by a shift in the metal oxide transition point and differences in the amount of current passing through the oxide. The MIT biosensor is useful for the detection of charged molecules, including macromolecules, such as proteins or nucleic acids as well as other types of particles, such as cells, bacteria, or viruses.

### CROSS-REFERENCE TO RELATED APPLICATION

This application claims the benefit of the filing date of U.S. Provisional Patent Application No. 61/943,015 filed Feb. 21, 2014, which is hereby incorporated by reference for all purposes as if set forth in its entirety herein.

### STATEMENT OF FEDERALLY SPONSORED RESEARCH OR DEVELOPMENT

This invention was made with Government support under contract HG000205 awarded by the National Institutes of Health (NIH). The Government has certain rights in this invention.

### TECHNICAL FIELD

The present invention pertains generally to biosensors for detection of analytes of interest, such as biomolecules, cells, and viruses.

### BACKGROUND

Detection of biological analytes is useful in various applications in biotechnology and personalized medicine. The analytes of interest may range from macromolecules, such as proteins and nucleic acids, to viruses and whole cells. One class of biosensors, electrical biosensors, show promise for point-of-care and other applications, but have had problems with stability and reliability, particularly biosensors relying on the detection of labeled molecules.

Thus, there remains a need for improved biosensors that can lower the cost of diagnostics in clinical settings and for use in research and drug discovery.

### SUMMARY

The invention relates to a novel biosensor, the metal-insulator transition (MIT) point biosensor, a non-expensive miniaturized device, having a small footprint and high sensitivity, which can measure molecular interactions or the presence of small amounts of molecules without the need for the molecules to be labeled. The sensor comprises a vanadium dioxide (VO₂) layer located between two metal measuring pads. The introduction of molecules of interest to the sensor surface results in changes in the oxide interface charge density that can be detected by a shift in the metal oxide transition point and differences in the amount of current passing through the oxide. The MIT biosensor is useful for the detection of charged molecules, including macromolecules, such as proteins or nucleic acids as well as other types of particles, such as cells, bacteria, or viruses.

In one aspect, a sensor is disclosed for detection of an analyte. The sensor includes two metal measuring pads, a vanadium dioxide (VO₂) layer in which the VO₂ layer is located between the two metal measuring pads, and a resistive heating mechanism electrically connected to the two metal measuring pads. The resistive heating element is configured to determine a change in the metal-insulator transition (MIT) point of the VO₂ layer therebetween in response to a binding of the analyte of interest to the sensor.

In some forms, the metal measuring pads may be gold, titanium, or platinum, for example.

In some forms, the sensor may further include a ligand immobilized on the surface of the VO₂ layer. This ligand may be for example, but not limited to, an antigen, an antibody, a hormone, a neurotransmitter, a receptor, an agonist, an antagonist, a substrate, an allosteric effector, an enzyme, a carbohydrate, a lectin, a drug, an inorganic molecule, or an organic molecule.

According to another aspect, a method is disclosed of using the sensor of the type described herein to detect the analyte of interest. The method includes the steps of contacting the VO₂ layer with a sample comprising the analyte of interest and measuring the metal-insulator transition (MIT) point of the VO₂ using the resistive heating mechanism. A detected change in the MIT point compared to a reference MIT point is used to indicate that the analyte of interest is bound to the sensor.

In some forms of the method, the method may further include the step of binding a ligand to the surface of the VO₂ layer. It is contemplated that the analyte of interest may bind to this ligand and that this analyte might be, for example, but not limited to, a molecule (including charged molecules or polar molecules; macromolecules such as proteins, nucleic acids, lipids, or carbohydrates), a cell (including eukaryotic or prokaryotic cells or cells from bacteria, protists, fungi, plants, or animals), a virus, or a particle. Further, the analyte may be unlabeled. It is contemplated that the ligand may be for example, but not limited to, an antigen, an antibody, a hormone, a neurotransmitter, a receptor, an agonist, an antagonist, a substrate, an allosteric effector, an enzyme, a carbohydrate, a lectin, a drug, an inorganic molecule, or an organic molecule.

In some forms, the molecular interactions may be detected between members of a binding pair.

According to yet another aspect, a microfluidic device is disclosed comprising one or more sensors of the type described herein.

In some forms, it is contemplated that the microfluidic device may include a plurality of the sensors and that these sensors may be organized in a parallel array.

If there are multiple sensors, then in another aspect, a method is disclosed for using the microfluidic device for multiplexed detection of analytes. This method includes the steps of binding a ligand to the surface of the VO₂ layer of each sensor in which a different ligand is bound to each sensor in the parallel array, contacting the VO₂ layer with a sample comprising one or more analytes of interest, and measuring the metal-insulator transition (MIT) point of the VO₂ for each sensor in the parallel array, wherein a change in the MIT compared to a reference MIT for a sensor indicates that an analyte of interest is bound to the sensor.

In some forms of this method, the ligand(s) may be, for example, but not limited to, an antigen, an antibody, a hormone, a neurotransmitter, a receptor, an agonist, an antagonist, a substrate, an allosteric effector, an enzyme, a carbohydrate, a lectin, a drug, an inorganic molecule, and/or an organic molecule. Further, the analytes of interest may be a molecule, a cell, a virus, and a particle (such as those described more explicitly above, for example). In some forms of the method, a different antibody is bound to each sensor in the parallel array.

Now, some other non-limiting advantages of MIT sensors are now presented for consideration. Again, these advantages are by way of example only and not all sensors or methods related thereto may realize these advantages.

A first advantage may be label-free detection. Label-free biosensors attempt to overcome the stability and reliability problems of biosensors relying on the detection of labeled molecules. Since it is a direct detection, only one antibody may be employed. Thus, the distortion of results due to cross-reactivity should be minimal relative to other sandwich immune based strategies for protein detection where two high quality antibodies for each target protein are needed. This technology is expected to outperform antibody-based array methods in terms of accuracy.

A second advantage may be high sensitivity. This biosensor can respond to not only surface charge accumulation, but also to polar molecules including possible antibody and antigen. As a result, the binding of just a few molecules to the sensor surface can cause a big change in the measured signal.

A third advantage may be that this technology offers the possibility for multiplexed biomarker detection. Multiplexing is a valuable feature to enable high throughput proteomics and genomics in large scale, and fabrication of an array of parallel MIT sensors in each microfluidic channel is another feature that improves the detection limit as the minimum concentration of target bio-molecule.

A fourth advantage may be that the sensors are relatively easy to fabricate and are inexpensive. Because one of the strengths of this technology is the low fabrication cost facilitating scalability, this technology can be easily multiplexed to detect different target proteins. MIT sensors may be fabricated in a parallel array, and different antibodies may be patterned on each sensor's surface.

A fifth advantage may be that the device may be miniaturized with a small footprint. In order to maintain the portability and low cost of this technology, the footprint of the readout instrumentation can be miniaturized. A low-noise CMOS (complementary metal oxide semiconductor) with a lock-in amplifier combined with the sensors can be custom designed.

A sixth advantage may be that this MIT sensor has no oxidation problems like other Si-based biosensors. Since vanadium dioxide is being used as the sensor surface, there is no risk of oxidation or the measured signal drift due to the chemical reactions like some of the silicon-based sensors.

These and still other advantages of the invention will be apparent from the detailed description and drawings. What follows is merely a description of some preferred embodiments of the present invention. To assess the full scope of the invention, the claims should be looked to as these preferred embodiments are not intended to be the only embodiments within the scope of the claims.

### BRIEF DESCRIPTION OF THE FIGURES

FIG. 1(a) illustrates schematics of MIT biosensors in which the measurement pads are illustrated as gold (Au), but could also be titanium or platinum. FIG. 1(a) shows (i) a bare MIT sensor, (ii) the MIT sensor with antibodies further attached to the surface of the oxide, and (iii) the MIT sensor in which antigens are further bonded to the attached antibodies. FIG. 1(b) schematically illustrates a parallel array of the MIT biosensors in a microfluidic channel for the multiplexed and label-free biomarker detection on a single chip.

FIG. 2(a) is a schematic of an MIT sensor. FIG. 2(b) is a typical current-voltage characteristic curve. FIG. 2(c) is a schematic of the experiment in which antigens are received on antibodies.

FIGS. 3 and 4 illustrate positive and negative control experiments, respectively, in which power and resistance of the material are measured at the point of switching for different steps in an experiment with biotinylated BSA or non-biotinylated BSA, respectively, and Streptavidin. Resistance and power are calculated and plotted at the metal-insulator transition point, by considering the shift in voltage and passing current at the metal-insulator transition point. Resistance is the darker line, while power is the lighter line. For the sake of clarity, it is noted that, in FIG. 3, the blue line is below the black line where the lines diverge. In FIG. 4, the lighter power line is above the darker resistance line in the “NBBSA” and “Wash NBBSA” steps but below the resistance line in the “Streptavidin” step.

FIG. 5(a) illustrates a schematic of a sensor. FIG. 5(b) is a schematic of the experimental procedure in which the sensor (i) is a virgin or bare sample, (ii) is a sample with a receptor added, and (iii) is a sample with target received on the receptor of (ii). FIG. 5(c) shows current-voltage plots of a representative sensor in both current and voltage controlled modes.

FIG. 6(a) shows current-voltage plots, measured upon sourcing a controlled voltage at different stages of the experiment done using biotinylated BSA and streptavidin. Inset is variation in the power required to switch, or power at the switching potential, at different stages of the experiment, corresponding to the legend in the main panel. FIG. 6(b) and inset are similar to (a), except that the experiment is performed using BSA and streptavidin. In FIG. 6(c), local temperature is measured at the center of a device (as schematically displayed in the inset) using a controlled current for a bare device and one treated with biotinylated BSA and streptavidin. “ON” and “OFF” labels correspond to the “ON” and “OFF” electrical switching events, as displayed in FIG. 6(c).

### DETAILED DESCRIPTION

The practice of the present invention will employ, unless otherwise indicated, conventional methods of chemistry, biochemistry, cell biology, molecular biology and recombinant DNA techniques, within the skill of the art. Such techniques are explained fully in the literature. See, for example, A. L. Lehninger, Biochemistry (Worth Publishers, Inc., current addition); Sambrook et al., Molecular Cloning: A Laboratory Manual (3rd Edition, 2001); Methods In Enzymology (S. Colowick and N. Kaplan eds., Academic Press, Inc.).

All publications, patents, and patent applications cited herein, whether supra or infra, are hereby incorporated by reference in their entireties for all purposes.

### I. DEFINITIONS

In describing the present invention, the following terms will be employed, and are intended to be defined as indicated below.

It must be noted that, as used in this specification and the appended claims, the singular forms “a”, “an”, and “the” include plural referents unless the content clearly dictates otherwise. Thus, for example, reference to “a ligand” includes a single ligand, a mixture of two or more ligands, and the like.

The term “about”, particularly in reference to a given quantity, is meant to encompass deviations of plus or minus five percent.

As used herein, the term “ligand” refers to a molecule that binds to another molecule, for example, an antigen binding to an antibody, a hormone or neurotransmitter binding to a receptor, a substrate or allosteric effector binding to an enzyme, or a carbohydrate binding to a lectin, and includes natural and synthetic biomolecules, such as proteins, polypeptides, peptides, nucleic acid molecules, carbohydrates, sugars, lipids, lipoproteins, small molecules, natural and synthetic organic and inorganic materials, synthetic polymers, and the like.

As used herein, the term “binding pair” refers to first and second molecules that specifically bind to each other. “Specific binding” of the first member of the binding pair to the second member of the binding pair in a sample is evidenced by the binding of the first member to the second member, or vice versa, with greater affinity and specificity than to other components in the sample. The binding between the members of the binding pair is typically noncovalent. Unless the context clearly indicates otherwise, the terms “ligand” and “target analyte” are used herein to refer to first and second members of a binding pair, respectively.

### II. MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, some preferred materials and methods are described herein.

The invention relates to a novel biosensor, the metal-insulator transition (MIT) point biosensor, a non-expensive miniaturized device, having a small footprint and high sensitivity, which can measure molecular interactions or the presence of small amounts of molecules without the need for the molecules to be labeled. The sensor comprises a vanadium dioxide (VO₂) layer located between two metal measuring pads. The introduction of molecules of interest to the sensor surface results in changes in the oxide interface charge density that can be detected by a shift in the metal oxide transition point and differences in the amount of current passing through the oxide. The MIT biosensor is useful for the detection of charged molecules, including macromolecules, such as proteins or nucleic acids as well as other types of particles, such as cells, bacteria, or viruses. The MIT biosensor also has the capability of processing a parallel array of sensors in microfluidic channels for multiplex detection of biomolecules on a single chip. Use of inexpensive materials and minimized fabrication steps lowers the cost of the MIT sensor dramatically. In addition, MIT biosensors do not have the oxidation or corrosion problems of most electrical biosensors. The MIT biosensor array has the potential to significantly lower the cost of diagnostics in clinical settings and may have substantial use in drug discovery.

It will be appreciated that VO₂ is a Mott insulator that undergoes an insulator-metal transition at roughly 340 K. During this phase transition, it undergoes a significant drop in resistivity and which is believed to be also closely accompanied by a structural phase transition from monoclinic to rutile crystal structure. This transition can be induced by different driving forces, including temperature, electric field, light, strain, doping, substrate, and so forth. Due to changes in resistance, crystal structure, and optical properties, this material has found various applications in different areas like neuromorphic computing, optics, non-volatile memory, micromechanics, and so forth. The driving mechanism behind the metal-insulator transition (MIT) in two terminal devices of VO₂ was under debate until recently, with competing claims supporting joule-heating and electric field as the driving mechanism. It was recently shown that the conductance switching caused by MIT is driven purely by joule heating, where the material undergoes a sufficient increase in temperature to induce a thermally driven transition.

This sensor works based on the change of metal-insulator transition (MIT) behavior by changing the oxide interfaces charge density. The metal-insulator transitions observed in correlated-electron materials provide an intriguing possibility. Some transition-metal oxides exhibit both metallic and insulating states, at a fixed carrier density, and can be switched between them by varying temperature, strain, and external electrical boundary conditions.

Some of the oxides such as vanadium dioxide (VO₂) have this property. A striking feature of these compounds arise from their first-order metal-insulator transition (MIT), in some conditions the system behaves as a half-filled metal state for VO₂, and some others the system adopts the insulating ground state, at which the resistance changes abruptly by several orders of magnitude. Others have used an electric-double-layer transistor (EDLT) technique involving an organic ionic liquid, which enables them to tune the surface charge density, on a VO₂ oxide layer as a bulk. M. Nakano et al, Collective bulk carrier delocalization driven by electrostatic surface charge accumulation, Nature 487, 459 (July 2012). In their study they showed that surface charge accumulation is accompanied by a collective lattice deformation along the c-axis direction, and resultant delocalization of previously localized electrons in the bulk VO₂ film, leading to a three-dimensional metallic ground state with high carrier density (‘proliferatively’ generated) throughout the film.

In this disclosure, attachment of biomolecules such as proteins or DNAs, or viruses, bacteria or any other particles with charge to the surface of the oxide are comtemplated such that the oxide serves as a sensor. The principle of operation is that, by changing the oxide interface charge density and its electrical boundary condition through reception of analytes (and/or receptors), a detectable change in metal-insulator transition (MIT) behavior of the oxide can result. The detection of attachment can be performed by looking at the change of switching voltage, which is due to the presence or interaction of biomolecules (or more, generally, receptors and analytes).

In order to further an understanding of the invention, a more detailed discussion is provided below regarding the MIT biosensor.

### EXAMPLES

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example I

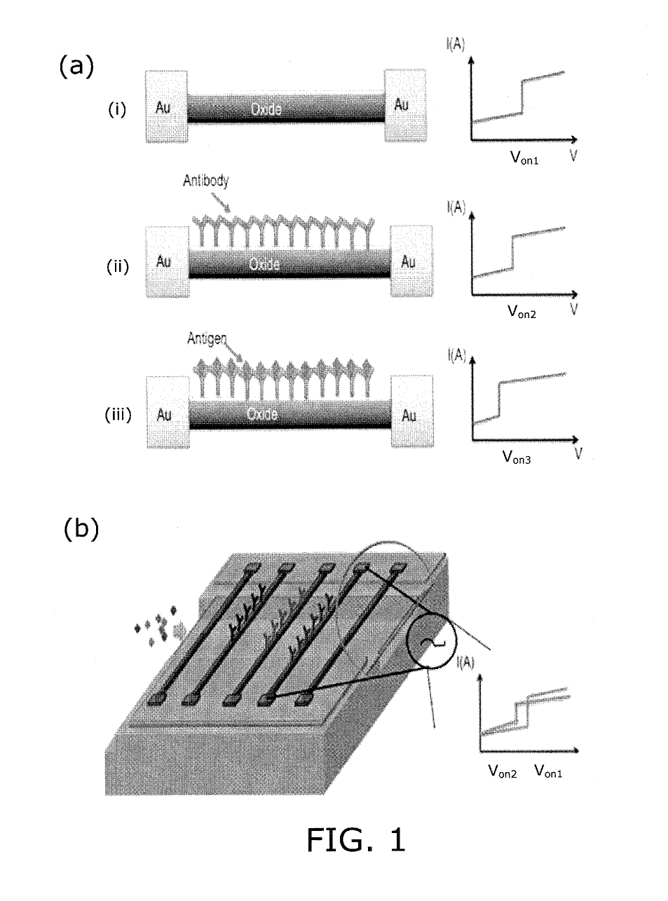

An MIT sensor structure is shown in FIGS. 1(a) and 1(b) in which the sensor structure comprises a vanadium dioxide (VO₂) layer (i.e., the oxide as labeled), which is located between two metal measuring pads (i.e., the gold (Au) as labeled but which might alternatively be, for example, titanium or platinum). These MIT sensors were fabricated using VO₂, which is a material that switches from a semiconducting to metallic phase at a certain electrical power. Vanadium dioxide undergoes a Mott transition from a semiconductor to a half filled metal at about 340K, which can be induced by joule heating by flowing current across the material using a pair of electrodes. This Mott transition temperature can be potentially lowered by electron doping of oxide, applying tension to the oxide, using different methods of fabrication, or some other methods.

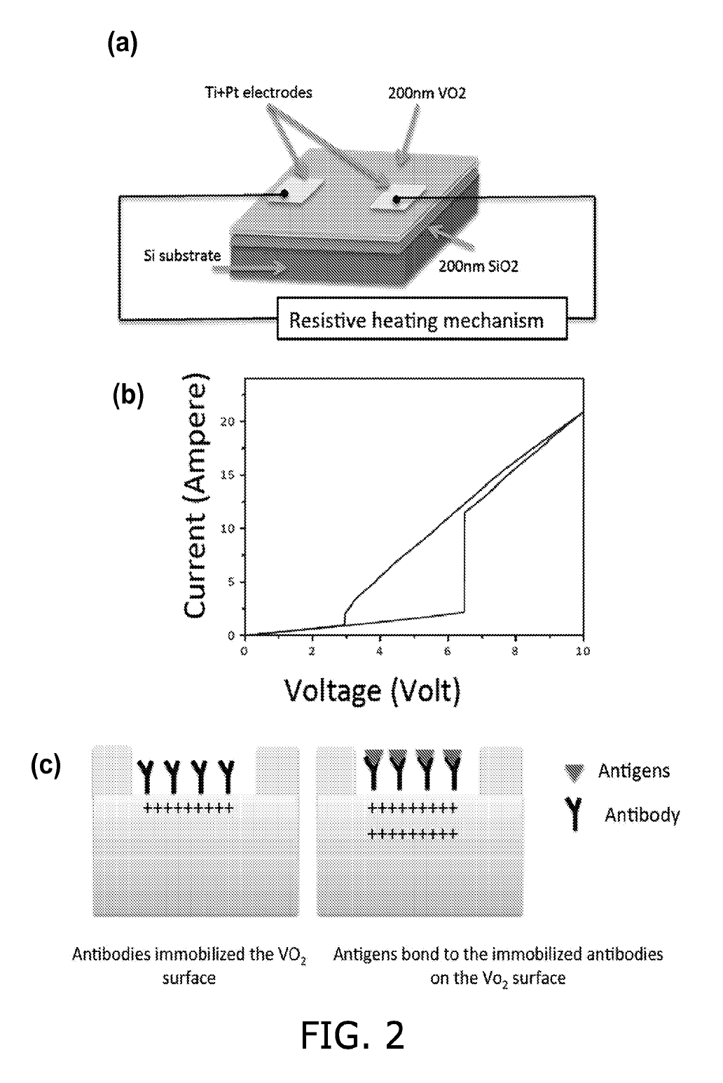

With additional reference being made to the schematic of FIG. 2(c), the sensing of this MIT sensor is premised on the measurable and observable change of the metal-insulator transition (MIT) point by altering the oxide interfaces charge density as the result of addition of one or more receptors and one or more analytes. Detection of this change can be done by measuring the change of a current-voltage curve (an I-V curve) of the sensor while the molecules of interest (i.e., the receptors or analytes) are introduced to the sensor surface as is schematically illustrated in FIGS. 1(a)(i) through 1(a)(iii) in which representative changes in the I-V curves are illustrated to the right side of each schematic. When the biomolecule with charges are attached to the oxide surface of the VO₂, the metal-oxide transition point is shifted as well as the passing current through the oxide. Since the transition point and the passing current both are different due to the attachment of the biomolecule(s), as a result the calculated electronic resistance (and power) at the metal-oxide transition point are different when receptors or analytes are attached.

The mechanism that results in this observable change is bulk delocalization by surface charge accumulation. By introducing charge to the oxide surface [either directly as in FIG. 1(a)(ii) and the left panel of FIG. 2(c) in which antibodies are attached to the oxide, or indirectly as in FIG. 1(a)(iii) and the right panel of FIG. 2(c) in which antigens are subsequently bound to the antibodies], charges on the surface are accumulated that create a dipole field at the surface which is subsequently shown to induce the insulator-metal Mott transition and which shifts the transition to a lower temperature. This transition at the surface also happens in the bulk of the VO₂, since the bulk is more stable in the metal phase when the surface is at the metal phase.

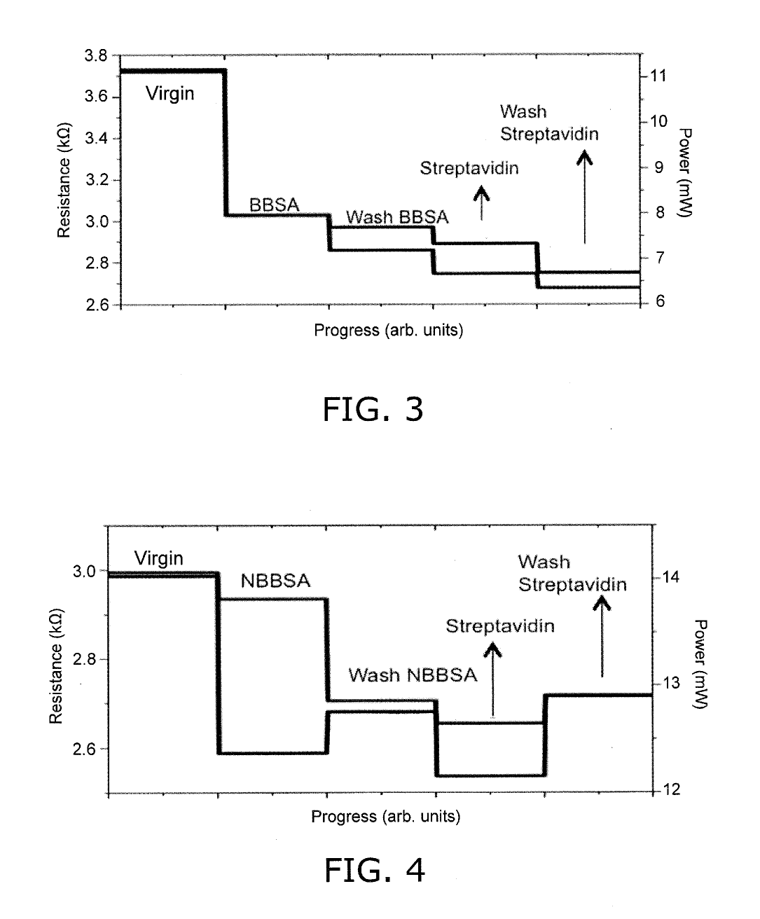

Experiments were run using these MIT sensors to establish that a biosensor can be made with VO₂ that responds to not only surface charge accumulation, but that also responds to polar molecules such as an antibody and antigen as are illustrated in FIGS. 3 and 4. In summary, a switching power change was observed upon treatment with biotinylated bovine serum albumin (BBSA or biotinylated BSA) and with subsequent treatment with streptavidin. Biotinylated BSA was used as the receptor and streptavidin as the target analyte. While the electrical power changes, the resistance of the semiconducting state stays constant. This implies that the temperature of the Mott transition itself has changed. It was also observed that this is specific to using streptavidin, which binds with biotinylated BSA as in the trial depicted in FIG. 3 and not to the non-biotinylated BSA (NBBSA) as in the trial depicted in FIG. 4, indicating that the MIT sensor has the desired specificity. A critical or threshold concentration of surface charges can create a Debye layer of charges at the surface that can induce a bulk delocalization and a 3D phase transformation in the VO₂. The increasing surface charges reduce the temperature of the transition. The surface charges induced by the charge carried by the receptors and the targets are believed to modify the temperature of the transition.

Now with additional reference to FIG. 2(a), a schematic of the fabricated biosensor is illustrated that was tested to determine the current-voltage (I-V) characteristics of the bare sensor. For this biosensor, the I-V curve typically resembles the one shown in FIG. 2(b). This I-V curve and the sudden discontinuity at just above 6 V is understood to be a result of the Mott transition that occurs in VO₂.

When biomolecules with charges are attached to the oxide surface, this metal-oxide transition point (i.e., the sudden discontinuity in electrical behavior) will be shifted as well as the passing current through the oxide. Since the transition point and the passing current both are different due to the attachment of biomolecules, as a result the calculated electronic resistance (and power) at the metal-oxide transition point is different. In fact, it dropped at about 70° C. when it was heated and came back to its initial value at about 60° C. when it was cooled, giving rise to the shown current-voltage curve in FIG. 2(b). The current and voltage at the switching point determine the power required to locally heat the material to make it switch resistance states.

In order to show the proof of concept for protein detection with MIT devices, the ability of the sensors to detect binding of biotin and streptavidin was tested in separate trials depicted in FIGS. 3 and 4. Biotinylated BSA (BBSA)-Streptavidin binding was chosen to demonstrate the effectiveness of device functionality. This binding has been extensively studied and is a well-understood process, and therefore can serve to model and characterize a system for protein interactions.

The procedure for the first trial, depicted stepwise in FIG. 3, was as follows: BBSA molecules, which act as receptor proteins, were injected on the sensor surface. This protein physically adsorbed and bound to the oxide surface. The BBSA solution was suspended in pure water at a concentration of 100 μg/ml. Then, the measurement well was dried out, and the switching metal-insulator transition (MIT) point was measured before and after washing it with water. Between every step, the measurement well was dried out, and all the measurements were performed in dry conditions. The reason to wash the well was to ensure that the changes in MIT point and its conductance were not spurious and due to molecules attaching to the surface. This resulted in no change in both beyond the noise level. Streptavidin was then injected into the channel. Streptavidin was resuspended in pure water at a concentration of 10 μg/ml. The binding of the streptavidin molecules to the BBSA resulted in a change in the metal-insulator transition (MIT) point of the sensor.

FIG. 3 shows the resistance and power differences over the course of this first trial. In FIG. 3, resistance and power are calculated at the metal-insulator transition point, by considering the shift in voltage and passing current at the metal-insulator transition point. As a control, water was injected into the well and the metal-insulator transition (MIT) point was measured after injection of streptavidin molecules. The change of metal-insulator transition (MIT) point after this washing step was insignificant to the extent that it was buried in noise. It showed that the measured conductance and metal-insulator transition (MIT) point were only modulated when a molecule is bound to the sensor, resulting in a change of conductance and MIT point. The measured metal-insulator transition (MIT) point changes were not due to unbound streptavidin molecules.

As illustrated in FIG. 4, a control trial was also performed to confirm that the changes in the conductance and MIT point were due to specific binding of streptavidin molecules to the biotinylated BSA molecules. For this second trial, regular non-biotinylated BSA (NBBSA) molecules were immobilized to the surface of the MIT sensor. The same procedure was followed as above for this control trial, except that all streptavidin molecules were removed from the well after washing the well (as they would not bind to the NBBSA since there was no biotin linked to the BSA molecules). The measured metal-insulator transition (MIT) point after the streptavidin washing step returned to its initial point before the injection of streptavidin molecules. This control experiment confirms that the change of metal-insulator transition (MIT) point was due to the specific protein binding.

As inducing surface charges was believed to affect the transition point, these tests with polar molecules like BSA and streptavidin serves as proof of concept that surface changes can alter bulk properties and measured electrical behavior of the VO₂. Again, FIG. 3 shows a plot of the power and resistance levels at the metal-insulator transition (MIT) point for different parts of the trial involving BBSA and streptavidin. The power level clearly goes down as the trial progressed. Each change in the power level indicates that there are charges sticking on to the surface of the material. The last step shows that, on the addition of streptavidin and subsequent washing, there was a significant change in the power level relative to the material at the stage where there was no streptavidin on it. This confirms functionality of the sensor.

Then, in the control experiment depicted in FIG. 4, the specificity of the sensor was established by using non-biotinylated BSA, which was not expected to let the streptavidin bind onto it. FIG. 4 clearly showed that, when the streptavidin is washed off, the power level reverts to that before the addition of streptavidin.

Thus, this example presents the fabrication and testing of metal-insulator transition (MIT) point biosensors for protein detection. The results of this example demonstrate the feasibility of using metal-insulator transition (MIT) point biosensors for detection of biomolecular interactions or any particle with charges. As already mentioned, electronic sensing using metal-insulator transition (MIT) point biosensor devices offers several advantages. These sensors are label free, small, fast, and low cost. The metal-insulator transition (MIT) point sensors can potentially be used for large-scale multiplexed cancer biomarker discovery. See, for example, the MIT sensor structure illustrated in FIG. 1(b). MIT biosensors can be designed and fabricated in a pixel array format as an integrated and localized biosensor with the possibility of arrays of hundreds of micro-sensors per square millimeter of a device. Because of the use of electrical detection, it is envisioned that these sensors could be applied in an integrated handheld system for point-of-care clinical diagnostics. This sensor can also be used for the label-free detection of DNA hybridization and label-free cell detection. This type of sensor does not have oxidation or correction problems that most silicon based biosensors have.

### Example II

In this example, a reduction is observed in the power required to switch the resistance state of the material upon treatment of the surface of lateral VO₂ devices with polar molecules. Using blackbody-emission temperature measurement, it is shown that this reduction in power is accompanied by a reduction in the switching temperature. BBSA-Streptavidin binding was again chosen to demonstrate the effect. As noted in Example I, this binding has been extensively studied and is a well-understood process, and therefore can serve to model and characterize a system for protein and receptor-target interactions. When the polar molecules bind to the surface and then to one another, it is believed that there is a net transfer of charge to the surface of VO₂ and hence a change in the transition temperature and the joule heating-driven switching current is observed. In addition, it is shown that when the species of molecules used in the experiment are not expected to bind to one another, there is much less, or negligible, net transfer of charge to the surface of VO₂, hence little or no change in the transition temperature.

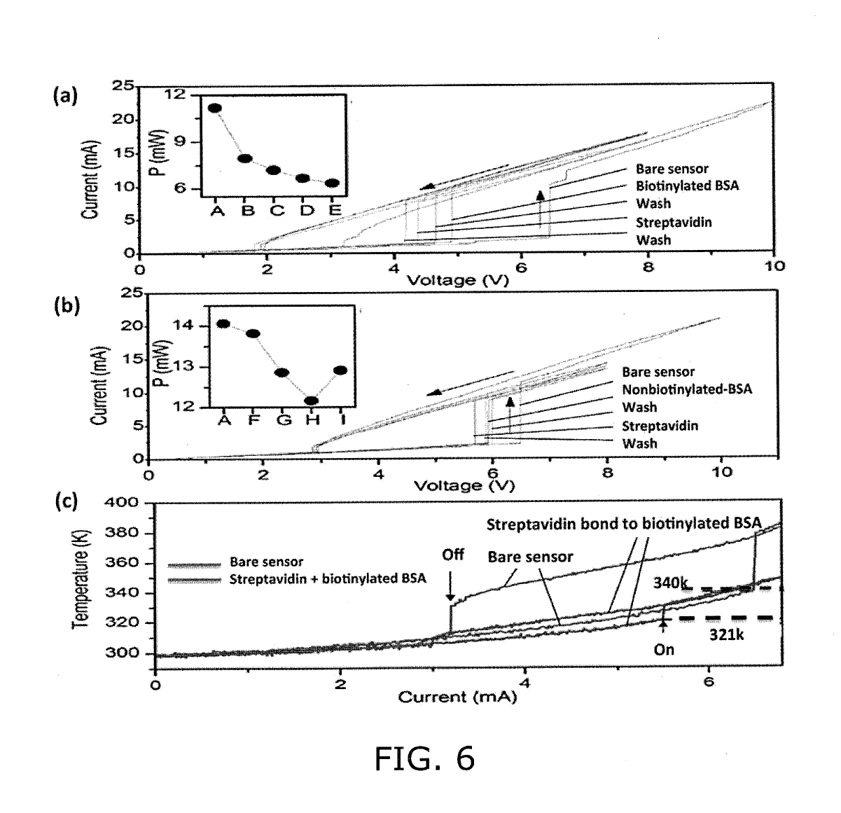

Although there is no comprehensive understanding of the role of BSA, biotin and streptavidin in inducing surface charges, there are several studies that have established some aspects of the microphysics of their behavior. Biotin-streptavidin interaction is one of the most widely used in bioconjugation chemistry, owing to the strong affinity and high specificity of the interaction. The presence of four binding sites on each streptavidin molecule makes it possible to link together biotin-tagged molecules or biotin-tagged molecules to a biotin functionalized surface. The functional groups of biotin that binds to streptavidin are oriented away from the BSA molecules. And streptavidin molecules also have specific functional groups that bind with biotin. Thus, the utility of BSA was in helping orient the functional groups of biotin that attaches with those in streptavidin and in helping the other species bind to the surface of VO₂, thereby yielding an array of polar molecules with a finite net directional orientation. Since biotin is a polar molecule, it was believed that the biotin bound to BSA would induce a net surface charge and hence a Debye layer on the surface of VO₂. This belief is also supported by the experiment described by FIGS. 6(a)-6(c), as noted later.

It is noted that there are variations in the electrical characteristics seen in FIGS. 6(a), 6(b), and 6(c). While the cause of this is being studied, it is emphasized that the example never compares two different devices. Instead, the evolution of the electrical characteristics of a single device upon treatment of polar organic molecules is compared to their preceding electrical characteristics, which is the metric for sensing. This variation can be partly attributed to the on-chip variation in the film resistance.

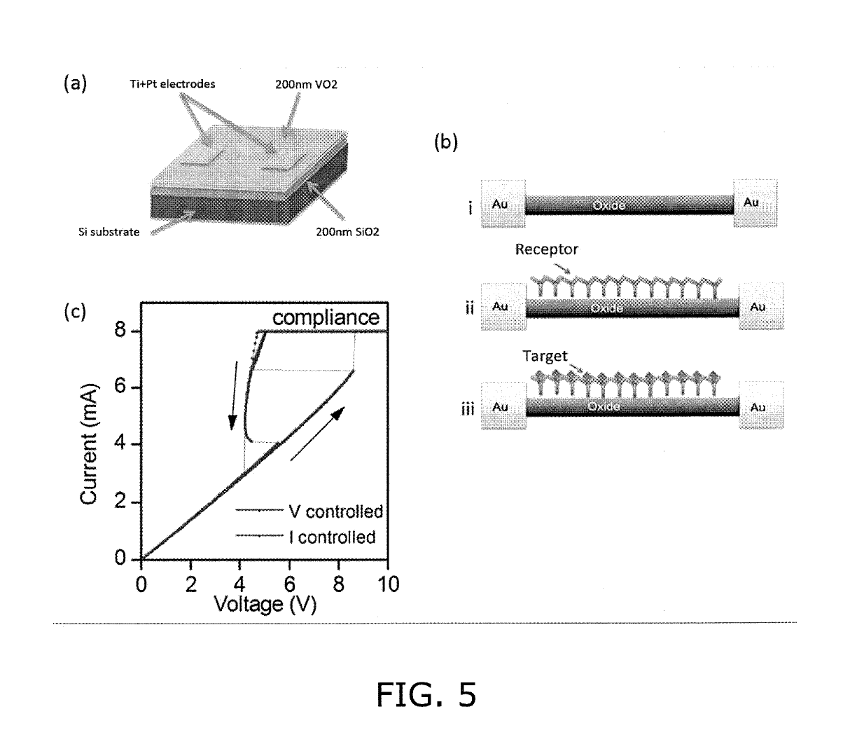

Electrical characterization of the effect was observed experimentally. FIG. 5(a) is a schematic of the device used, with a 200 nm blanket VO₂ film and 8 μm wide electrodes with 16 μm in between, forming the channel.

FIG. 5(b) is a schematic of the experiment that is the basis for this study which is similar to that found in Example I. Biotin and BSA (biotinylated BSA, suspended in pure water at a concentration of 100 μg/ml) were deposited on VO₂, followed by streptavidin (suspended in pure water at a concentration of 10 μg/ml).

Now with further reference to FIG. 6, FIG. 6(a) illustrates the experiment corresponding to the schematic of FIG. 5(b). The deposition of biotinylated BSA is observed to reduce the switching current. The addition of streptavidin is also observed to have caused a further lowering in the switching current. The surface was washed after each deposition process to make sure that we removed all molecules that were not bound to the surface that might have been randomly oriented, for, the nature of the bound molecules determines the nature of the Debye layer induced. In this example, washing the surface provided a stronger effect on the switching current, probably because washing leaves behind only those molecules aligned and held by their polar and functional natures, hence increasing the net charge from the molecules contributing to the Debye layer.

The inset in FIG. 6(a) is a plot of the power at the switching current. This inset shows a monotonic decrease in power required for switching. It is noted that the procedure described for the trial run in FIG. 6(a) generally corresponds to the first trial in Example I and much of the data correlates comparatively.

Then, an identical experiment was performed using BSA instead of biotinylated BSA, as is illustrated in FIG. 6(b). Streptavidin does not bind to BSA as strongly as it does with biotin, and so a negligible effect upon addition of streptavidin was observed as evidenced by the data in FIG. 6(b). In particular, the inset in FIG. 6(b) shows that upon addition of streptavidin (state H), the power required to switch alters (or, the temperature of switching alters). But the material reverts to a state similar to that before addition of streptavidin (state G) upon washing of the sample (state I). This indicates that streptavidin did not bind to the surface, and it is suspected that the effect seen upon its deposition was only due to unbound molecules on the surface, inducing a net charge and a Debye layer. This is confirmation to the specificity of the sensing mechanism. Since streptavidin-biotin interactions occur through specific chemical groups in the oriented polar molecules, the experiment summarized in FIG. 6(b) supports the notion that there is a Debye layer that is induced because of polar molecules with a finite net orientation on the surface of VO₂.

If the deposited molecules could be removed, then the effect of such an operation on the behavior of the film could be examined. Removing the polar molecules from the VO₂ surface is possible through several chemical processes by changing the environmental pH, however such techniques caused damage to the VO₂ film including peel-off, evidencing that the binding is very strong. In case this was possible, it would not be expected to see a change in the films behavior (in specific, the film reverting to the virgin state) immediately upon removal of the deposited molecules, as against the film's behavior in the presence of these molecules. This follows from the electro-chemical characterization performed by others using ionic liquid gating. Interestingly, to be able to retrieve the virgin state, the film must acquire a Debye layer with inverted charge layers relative to that which caused the change in resistance to begin with. This is far easier to explore with a controllable ionic gating (as has been done elsewhere), while the aim here was to explore the sensing ability of the material.

A change in power at the switching current is believed to be an indication of a change in the transition temperature, since the switching is driven by joule-heating. But this reduction in power could also be caused by other factors, for instance, inhomogeneity in the film, possibly caused by addition of multiple surface layers, as in this experiment.

To determine if the temperature of transition really changed, in-situ blackbody emission measurements were used to directly measure local temperature during switching. The “bare sensor” curve in FIG. 6(c) is a plot of the local temperature measured over a spot of 1-2 μm at the center of a device, in between the electrodes as a controlled current was ramped through the switching threshold of the device. The temperature at the switching threshold, identifiable by the abrupt redistribution in local temperature, is 340 K, roughly the same as the temperature required for a thermally driven transition. This plot reasserts that local joule-heating is sufficient to completely account for the conductance-switching.

The local temperature was then measured with the same technique on a device with identical geometry, and electrical behavior, which was treated with biotinylated BSA and streptavidin in FIG. 6(c). The temperature corresponding to the conductance switching (again, seen as an abrupt jump, marked “ON”) is about 321 K, well below 340 K. This is direct evidence suggesting that the expected changes in switching current observed in FIGS. 6(a) and 6(b) were also accompanied by a change in the Mott transition temperature.

Since this is a surface sensitive technique and temperature measured on a bare surface is being compared to that on a treated surface, a temperature gradient across the layer of deposited/treated molecules could result in measurement of a slightly lower temperature than that at the surface of VO₂. While possible artifacts like this are acknowledged, the significant change in measured transition temperature is believed to be supported by other evidence (for example, in FIGS. 6(a) and 6(b)) that allow FIG. 6(c) to qualitatively justify the conclusion that the transition temperature did change.

While others have allegedly observed a structural phase transition throughout the thin-film upon MIT due to surface charge accumulation; here, it is shown that structural composition averaged over the thickness of the film is unaffected with lowering in the transition temperature. It is acknowledged the surface charges, in this case, by themselves did not induce an MIT and we needed additional energy from joule-heating to induce MIT (FIG. 6(c)), and possibly a structural transition upon MIT. Nonetheless, this may help to place bounds on the validity of prior example.

A physical understanding of the altering of phase transition temperature in VO₂ due to surface charges was the hypothesis of surface charge accumulation causing a bulk carrier delocalization and effectively getting the material out of its Mott insulator state once the surface charges exceed a threshold concentration. But this theory was brought under question by the evidence that the oxidation state of vanadium and the oxygen content in VO₂ change when accumulation of surface charges caused a significant reduction in the transition temperature to induce the MIT.

But in this Example, it can be seen that there is reduction in the transition temperature (although the charges are not sufficient to cause a metal-insulator transition by themselves) with no change in the oxidation state of the material. Hence, this Example shows that a chemical change by itself is not responsible for the mere lowering of the transition temperature, although such a chemical change might be the cause of or accompanies the charge-induced metal-insulator transition as others have discussed.

In conclusion, it has been shown that deposition of specific polar species on surface of a Mott insulator like VO₂ alters the transition temperature sufficient enough to enable the detection of the polar species. It has been observed that the switching current of the devices reduces upon treatment with polar molecules and is further observed that it is accompanied by a reduction in the switching temperature. It has been shown that the sensor can detect charged species with specificity. It is also shown that vanadium's oxidation state or the material's structure is not altered during charge sensing. This result shows promise for this effect as a sensing mechanism, especially for biological and physical applications, while this result also adds to the interesting puzzles and possible solutions surrounding phase transitions in VO₂.

It should be appreciated that various other modifications and variations to the preferred embodiments can be made within the spirit and scope of the invention. Therefore, the invention should not be limited to the described embodiments. To ascertain the full scope of the invention, the following claims should be referenced.

What is claimed is:

1. A sensor comprising:
two metal measuring pads; and
a vanadium dioxide (VO₂) layer, wherein the VO₂ layer is located between the two metal measuring pads; and
a resistive heating mechanism electrically connected to the two metal measuring pads and configured to determine a change in the metal-insulator transition (MIT) point of the VO₂ layer therebetween in response to a binding of an analyte of interest to the sensor.

2. The sensor of claim 1, wherein the metal measuring pads comprise a metal selected from the group consisting of Au, Ti, and Pt.

3. The sensor of claim 1, further comprising a ligand immobilized on the surface of the VO₂ layer.

4. The sensor of claim 3, wherein the ligand comprises an antigen, an antibody, a hormone, a neurotransmitter, a receptor, an agonist, an antagonist, a substrate, an allosteric effector, an enzyme, a carbohydrate, a lectin, a drug, an inorganic molecule, or an organic molecule.

5. A method of using the sensor of claim 1 to detect the analyte of interest, the method comprising:
contacting the VO₂ layer with a sample comprising the analyte of interest, and
measuring the metal-insulator transition (MIT) point of the VO₂ using the resistive heating mechanism, wherein a change in the MIT point compared to a reference MIT point indicates that the analyte of interest is bound to the sensor.

6. The method of claim 5, further comprising binding a ligand to the surface of the VO₂ layer.

7. The method of claim 6, wherein the analyte of interest binds to the ligand.

8. The method of claim 6, wherein the ligand comprises an antigen, an antibody, a hormone, a neurotransmitter, a receptor, an agonist, an antagonist, a substrate, an allosteric effector, an enzyme, a carbohydrate, a lectin, a drug, an inorganic molecule, or an organic molecule.

9. The method of claim 5, wherein the analyte of interest is selected from the group consisting of a molecule, a cell, a virus, and a particle.

10. The method of claim 9, wherein the molecule is a charged molecule or a polar molecule.

11. The method of claim 9, wherein the molecule is a macromolecule.

12. The method of claim 11, wherein the macromolecule is a protein, nucleic acid, lipid, or carbohydrate.

13. The method of claim 9, wherein the cell is eukaryotic or prokaryotic.

14. The method of claim 13, wherein the cell is from bacteria, protists, fungi, plants, or animals.

15. The method of claim 5, wherein the analyte is unlabeled.

16. The method of claim 6, wherein molecular interactions are detected between members of a binding pair.

17. A microfluidic device comprising at least one sensor of claim 1.

18. The microfluidic device of claim 17, comprising a plurality of the sensors.

19. The microfluidic device of claim 18, wherein the plurality of the sensors is organized in a parallel array.

20. A method of using the microfluidic device of claim 19 for multiplexed detection of analytes, the method comprising:
binding a ligand to the surface of the VO₂ layer of each sensor, wherein a different ligand is bound to each sensor in the parallel array;
contacting the VO₂ layer with a sample comprising one or more analytes of interest, and
measuring the metal-insulator transition (MIT) point of the VO₂ for each sensor in the parallel array, wherein a change in the MIT compared to a reference MIT for a sensor indicates that an analyte of interest is bound to the sensor.

21. The method of claim 20, wherein at least one ligand comprises an antigen, an antibody, a hormone, a neurotransmitter, a receptor, an agonist, an antagonist, a substrate, an allosteric effector, an enzyme, a carbohydrate, a lectin, a drug, an inorganic molecule, or an organic molecule.

22. The method of claim 20, wherein at least one analyte of interest is selected from the group consisting of a molecule, a cell, a virus, and a particle.

23. The method of claim 20, wherein a different antibody is bound to each sensor in the parallel array.
